(19) United States
(12) Patent Application Publication
(10) Pub. No.: US 2018/0189992 A1
(43) Pub. Date: Jul. 5, 2018

(54) SYSTEMS AND METHODS FOR GENERATING AN ULTRASOUND MULTIMEDIA PRODUCT

(71) Applicant: Clarius Mobile Health Corp., Burnaby (CA)

(72) Inventors: Laurent PELISSIER, North Vancouver (CA); Kris DICKIE, Vancouver (CA); Clark VAN OYEN, New Westminster (CA); Shirley YIN, Vancouver (CA)

(21) Appl. No.: 15/398,650

(22) Filed: Jan. 4, 2017

(51) Int. Cl. G06T 11/60; A61B 8/08; G06T 7/00; G06K 9/32

(52) U.S. Cl. CPC G06T 11/60; A61B 8/0866; G06F 3/0482; G06T 7/0014; G06K 9/3258; A61B 8/5292

(57) ABSTRACT

The present embodiments relate generally to systems and methods for generating a multimedia product. The embodiments may include: identifying a plurality of ultrasound media items from a fetal ultrasound scan, where each of the plurality of ultrasound media items have different respective attributes; and applying a theme to the plurality of ultrasound media items to generate the multimedia product. The theme may include an effect to be applied to at least one ultrasound media item of the plurality of ultrasound media items, and the applying may include adapting one of: the effect, and the attribute of the at least one ultrasound media item, to the other.

### FIELD

The present disclosure relates generally to ultrasound imaging, and in particular, to systems and methods for generating an ultrasound multimedia product.

### BACKGROUND

Ultrasound is commonly used in medical examinations. For example, obstetrics examinations typically involve ultrasound scans of a fetus. These scans produce media items (e.g., images, videos, cineloops) interpreted by medical professionals to assess the development of the fetus. Since these scans usually provide the first images of an unborn baby, the media items may carry particular emotional meaning for parents.

Given the nature of ultrasound media items, it may be difficult for parents to interpret them. To make the ultrasound media items more digestible, some traditional systems allow users to manually select the media items from an obstetrics examination for the purpose of combining with audio, visual or text effects to generate a multimedia product. Using these traditional systems, the multimedia product may be written to physical media (such as a Compact Disc-Recordable (CD-R) or a Digital Video Disc (DVD)) or made available online.

Using these traditional methods to generate an ultrasound multimedia product is cumbersome. Manual selection of desirable media items, audio, and/or text overlays may be required prior to a multimedia product being generated. In some cases, manual effort is also required to transfer the media items to a computer where the multimedia product can be generated.

There is thus a need for improved systems and methods for generating an ultrasound multimedia product. The embodiments discussed herein may address and/or ameliorate at least some of the aforementioned drawbacks identified above. The foregoing examples of the related art and limitations related thereto are intended to be illustrative and not exclusive. Other limitations of the related art will become apparent to those of skill in the art upon a reading of the specification and a study of the drawings herein.

### BRIEF DESCRIPTION OF THE DRAWINGS

Non-limiting examples of various embodiments of the present disclosure will next be described in relation to the drawings, in which:

FIG. 1 is a user interface showing example ultrasound media items from an obstetrics examination, in accordance with at least one embodiment of the present invention;

FIG. 2 shows a simplified view of a timeline for a generated multimedia product, in accordance with at least one embodiment of the present invention;

FIG. 3 shows example relationships between the metadata of ultrasound media items and anatomical features, and also example relationships between anatomical features and available options for various effects, in accordance with at least one embodiment of the present invention;

FIGS. 4-5 are example screenshots of an ultrasound media product that have effects combined with ultrasound media items, in accordance with at least one embodiment of the present invention;

FIG. 6 is a flowchart diagram showing steps of a method of selecting an ultrasound media item for inclusion into a multimedia product, in accordance with at least one embodiment of the present invention;

FIG. 7 is an example illustration of cineloops having different lengths that are to be adapted when combined with an effect with a standard duration, in accordance with at least one embodiment of the present invention;

FIGS. 8A-8C are a sequence of example screenshots at various points in time of an ultrasound media product, in accordance with at least one embodiment of the present invention; and

FIG. 9 is a block diagram for a system of generating an ultrasound media product, in accordance with at least one embodiment of the present invention.

### DETAILED DESCRIPTION

In a first broad aspect of the present disclosure, there is provided a method of generating a multimedia product. The method includes: identifying a plurality of ultrasound media items from a fetal ultrasound scan, wherein each of the plurality of ultrasound media items have different respective attributes; and applying a theme to the plurality of ultrasound media items to generate the multimedia product, the theme comprising an effect to be applied to at least one ultrasound media item of the plurality of ultrasound media items; wherein the applying comprises adapting one of: the effect, and the attribute of the at least one ultrasound media item, to the other.

In some embodiments, the different respective attributes correspond to viewable anatomical features, and the method further includes, prior to applying the theme, detecting an anatomical feature viewable on the at least one ultrasound media item. In some embodiments, the anatomical feature is selected from the group consisting of: heart, arm, leg, face, head, brain, spine, kidney, liver, sexual organ, digits, belly, feet and hand. In some embodiments, the effect includes multiple options, and the adapting comprises selecting an option from the multiple options based on the anatomical feature.

In some embodiments, the plurality of ultrasound media items include metadata, and the detecting includes determining the anatomical feature viewable on the at least one ultrasound media item based on the metadata associated with the at least one ultrasound media item. In some embodiments, the metadata includes measurements, and the determining the anatomical feature is performed based on a type of measurement associated with the at least one ultrasound media item. In some embodiments, the metadata includes annotations, and the determining the anatomical feature is performed based on text present in the annotations associated with the at least one ultrasound media item.

In some embodiments, the detecting the anatomical feature is performed by comparing images in the plurality of ultrasound media items to a template image of the anatomical feature. In some embodiments, the template image is derived from a plurality of pre-categorized images showing the anatomical feature.

In some embodiments, the plurality of ultrasound media items includes a plurality of cineloops, and the different respective attributes of the plurality of ultrasound media items correspond to respective lengths of each cineloop. In some embodiments, the effect includes a duration of the effect. In some embodiments, the at least one ultrasound media item includes at least one cineloop, and the adapting comprises modifying one of: the length of the at least one cineloop and the duration of the effect.

In some embodiments, the effect is selected from the group consisting of: audio, animation, text, images, frames and borders. In some embodiments, the fetal ultrasound scan is performed for an obstetrics examination.

In some embodiments, prior to the identifying the plurality of ultrasound media items, the method further includes: displaying a user interface for displaying the plurality of ultrasound media items, the user interface providing a user-selectable option for generating the multimedia product; and receiving input that selects the user-selectable option for generating the multimedia product.

In another broad aspect of the present disclosure, there is provided a server including at least one processor and at least one memory storing instructions for execution by the at least one processor, wherein when executed, the instructions cause the at least one processor to: identify a plurality of ultrasound media items from a fetal ultrasound scan, wherein each of the plurality of ultrasound media items have different respective attributes; and apply a theme to the plurality of ultrasound media items to generate the multimedia product, the theme comprising an effect to be applied to at least one ultrasound media item of the plurality of ultrasound media items; wherein the applying comprises adapting one of: the effect, and the attribute of the at least one ultrasound media item, to the other.

In some embodiments, the different respective attributes correspond to viewable anatomical features, and the instructions further cause the processor to, prior to applying the theme, detect an anatomical feature viewable on the at least one ultrasound media item.

In some embodiments, the plurality of ultrasound media items comprises a plurality of cineloops, and the different respective attributes of the plurality of ultrasound media items correspond to respective lengths of each cineloop.

In another broad aspect of the present disclosure, there is provided a computing device comprising at least one processor and at least one memory storing instructions for execution by the at least one processor, wherein when executed, the instructions cause the at least one processor to: identify a plurality of ultrasound media items from a fetal ultrasound scan, wherein each of the plurality of ultrasound media items have different respective attributes; and apply a theme to the plurality of ultrasound media items to generate the multimedia product, the theme comprising an effect to be applied to at least one ultrasound media item of the plurality of ultrasound media items; wherein the applying comprises adapting one of: the effect, and the attribute of the at least one ultrasound media item, to the other.

In another broad aspect of the present disclosure, there is provided a computer readable medium storing instructions for execution by at least one processor, wherein when the instructions are executed by the at least one processor, the at least one processor is configured to: identify a plurality of ultrasound media items from a fetal ultrasound scan, wherein each of the plurality of ultrasound media items have different respective attributes; and apply a theme to the plurality of ultrasound media items to generate the multimedia product, the theme comprising an effect to be applied to at least one ultrasound media item of the plurality of ultrasound media items; wherein the applying comprises adapting one of: the effect, and the attribute of the at least one ultrasound media item, to the other.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

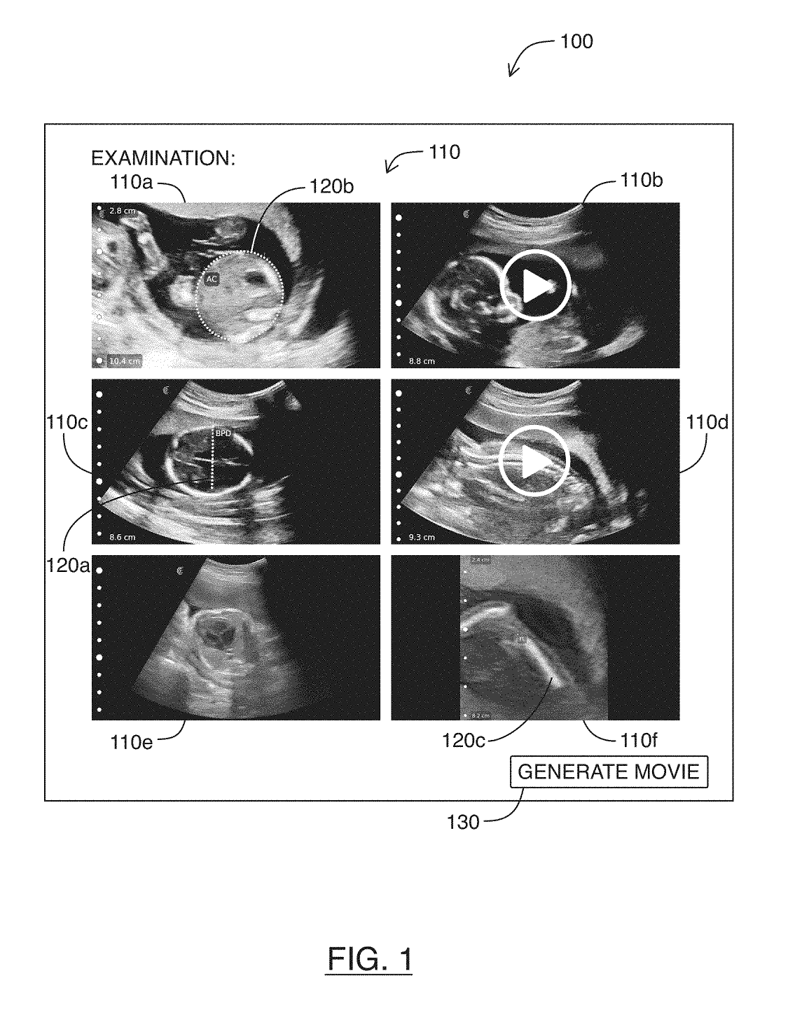

Referring to FIG. 1, shown there generally as 100 is a user interface illustrating example ultrasound media items from an obstetrics examination, in accordance with at least one embodiment of the present invention. In some embodiments, the fetal ultrasound scan is performed for a regular obstetrics examination during a pregnancy. As will be understood by persons skilled in the art, there are several standard views of a fetus that may be considered part of a standard ultrasound scan during pregnancy. These views may allow medical professionals to determine whether the growth of the unborn baby is proceeding as expected. During the scans, various media items may be obtained.

In FIG. 1, an example user interface showing these various media items 110 is provided. Particularly, six example ultrasound media items 110 are shown: an image 110a showing the abdomen of the unborn baby, a cineloop 110b showing a side profile of the unborn baby with its face viewable, an image 110c showing the head of the unborn baby, a cineloop 110d showing the spine of the unborn baby, an image 110e showing the fetal heart of the unborn baby, and an image 110f showing the femur of the unborn baby.

As part of an obstetrics examination, the medical professional may perform various measurements based on the ultrasound media items 110 obtained. These measurements may, for example, assist the medical professional with dating a fetus. As illustrated, measurements may be performed on the ultrasound image 110a showing the abdomen of the unborn baby to obtain an abdominal circumference (AC) measurement. Similarly, in the image 110c showing the head, a biparietal diameter (BPD) measurement may typically be taken to determine the diameter between the two sides of the head. Further, in the image 110f showing the femur, a femur length (FL) measurement may be taken to determine the length of the bone. As the femur is the longest bone in the body, the FL measurement may help the medical professional to assess the longitudinal growth of the fetus.

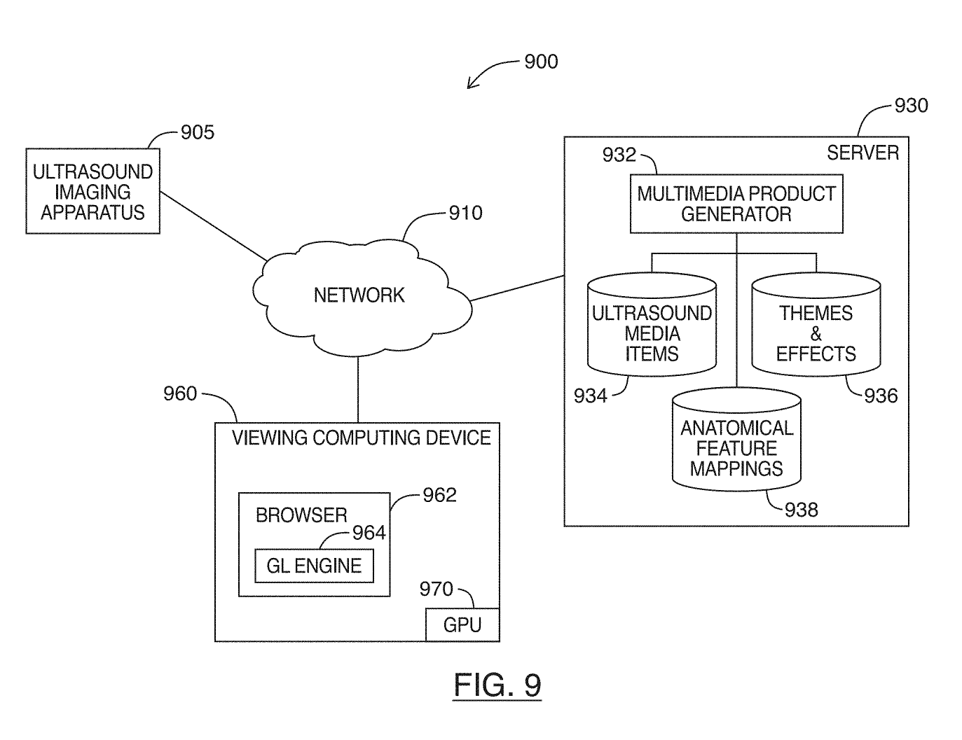

On their own, the various media items 110 shown in FIG. 1 may be difficult to understand for parents of the unborn baby who are unfamiliar with reading ultrasound media. To assist with creating a multimedia product that is more easily understandable, the user interface 100 may provide a user-selectable option 130 for generating the multimedia product (shown as a button labeled “Generate Movie” in FIG. 1). Upon receiving input that selects the user-selectable option 130 for generating the multimedia product, a corresponding computing device generating the user interface 100 may initiate execution of the methods of generating the multimedia product discussed below. Referring briefly to FIG. 9, the computing device for generating the user interface 100 may be part of the ultrasound imaging apparatus 905, and the initiation of the methods for generating the ultrasound media product may involve communicating with a remote server 930. Additional details related to the components of system 900 are discussed below.

Referring back to FIG. 1, upon selection of the user-selectable option 130, a method of generating a multimedia product may be performed. The methods of generating a multimedia product discussed here may include: identifying a number of ultrasound media items from a fetal ultrasound scan; for example, as may be shown in the examination of FIG. 1. The method may next apply a theme to the identified ultrasound media items to generate the multimedia product by imposing an order in which the media items 110 are to be displayed. In addition, the theme may involve applying various effects to the media items 110.

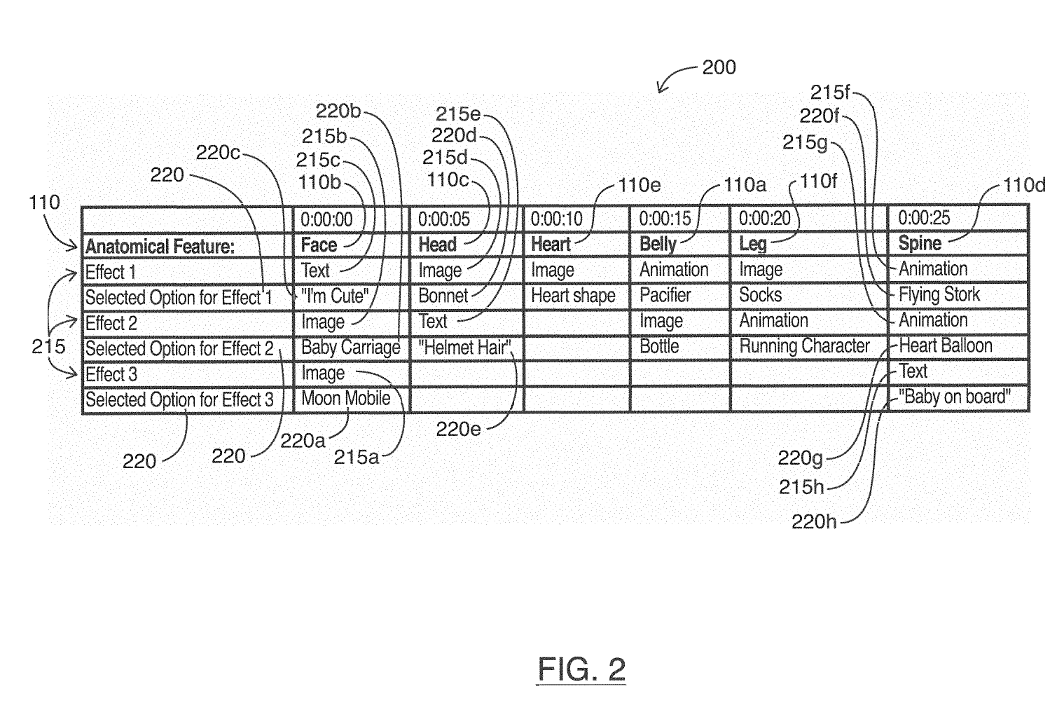

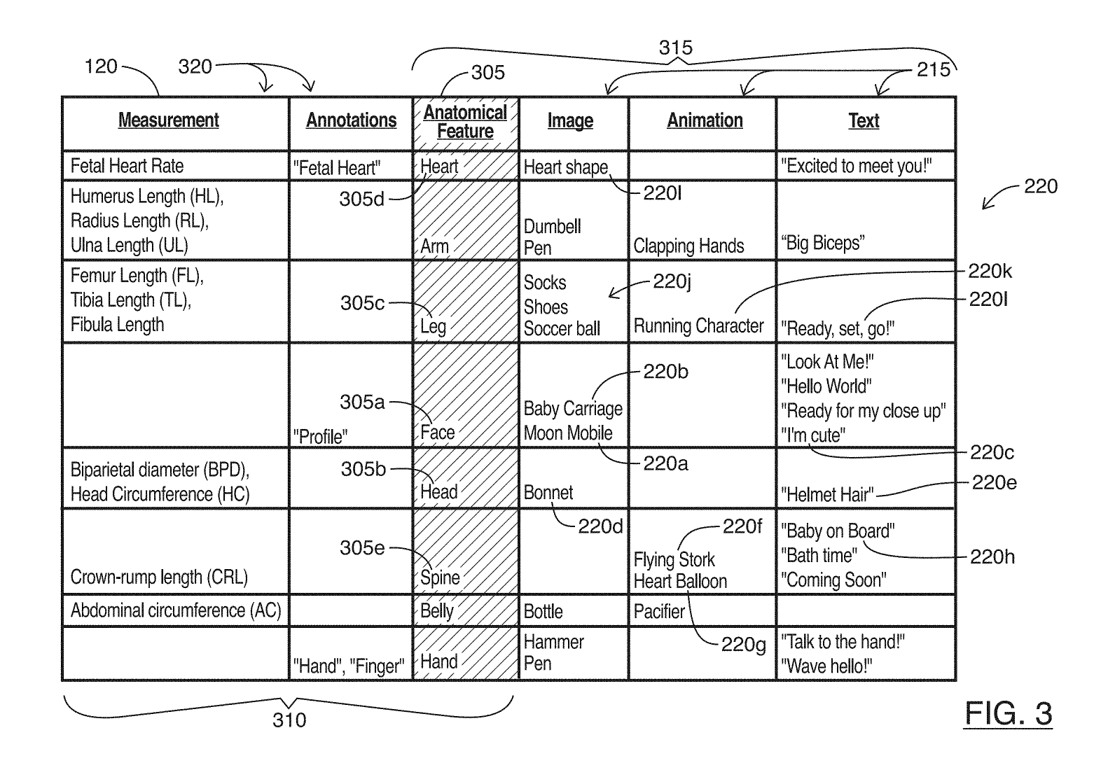

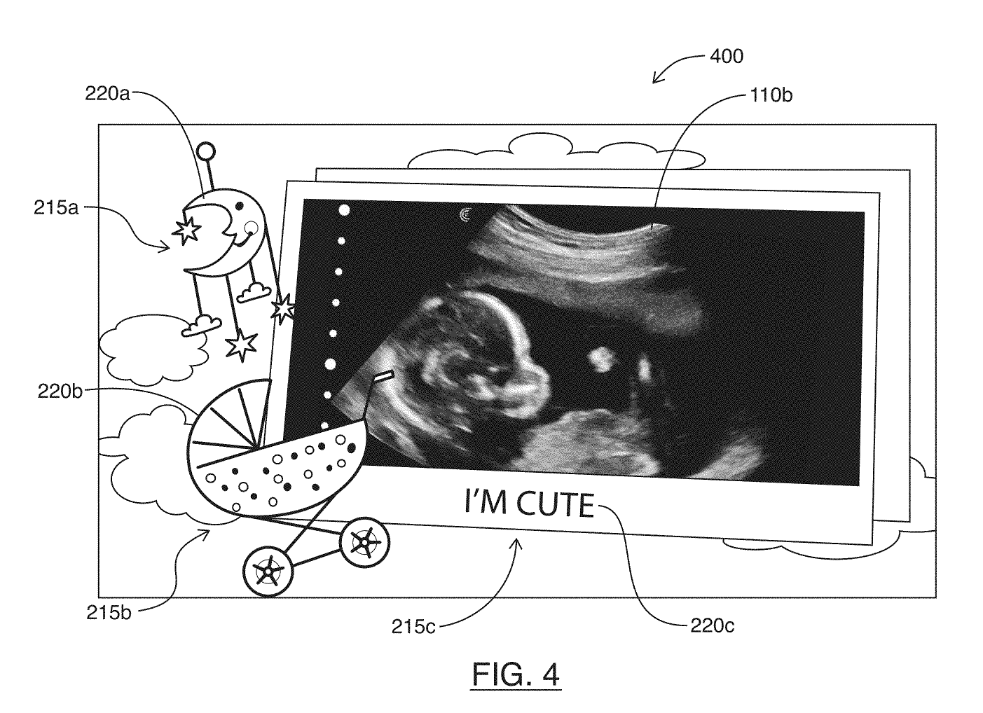

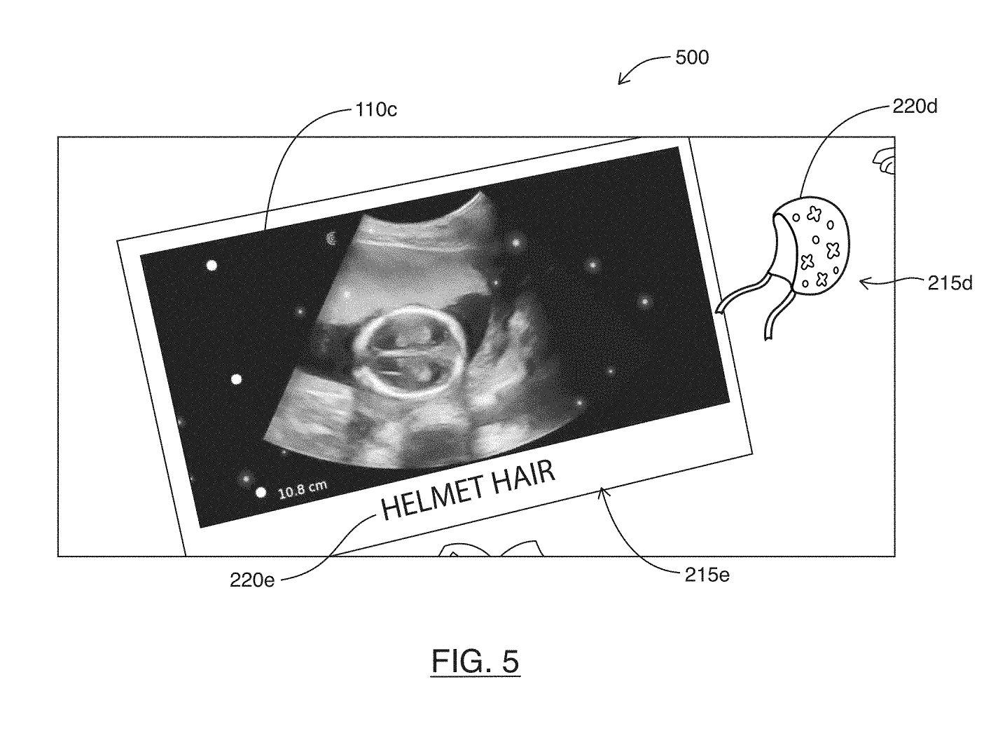

Referring to FIG. 2, shown there generally as 200 is an example simplified view of a timeline for a generated multimedia product, in accordance with at least one embodiment of the present invention. The timeline view is provided to illustrate various effects being applied to different media items 110. As illustrated, the timeline view is provided in a simple table format where the timestamps are provided on the top row, and corresponding media items 110 that are being displayed during the time period between successive timestamps are shown in the second row. The one or more effects 215 associated with a given media item 110 when it is being displayed are then shown in successive rows below a given media item 110. For each effect 215, an example option 220 is shown below the listed effect. In discussing FIG. 2, reference will simultaneously be made to the mappings in FIG. 3, and the various screenshots shown in FIGS. 4-5.

In various embodiments, applying a theme may include adapting a given effect to an attribute of a media item 110, and/or conversely, adapting an attribute of a media item 110 to the to the given effect. Different media items 110 may have different respective attributes. For example, an anatomical feature viewable in a given media item 110 may be considered to be an attribute of the media item 110. In other examples, the ultrasound media items 110 may be a number of different cineloops, and the attributes of the ultrasound media items 110 may be the respective lengths of the cineloops.

In some embodiments, the act of adapting an effect to an attribute of the media item 110 may include selecting an option for the effect to be used with a media item 110. As shown in FIG. 2, the applied theme may configure the media item 110b where the baby's profile and face is viewable to start play at the timestamp “0:00:00”. The theme may also be configured to use an image-type effect 215a with the media item 110b. However, there may be many different image options available to be used.

To adapt the image effect 215a to the media item 110b, an option for the image effect 215a may be selected based on the anatomical feature that is viewable in the media item 110b. In some embodiments, this may be performed via a lookup in a database or other suitable data structure where anatomical features typically viewable in ultrasound media items 110 are mapped to predetermined options 220 for certain effects 215. While these options 220 are predetermined at the particular time a lookup is made, it may be possible for the options 220 mapped to the effects 215 to be continually updated (e.g., as new options 220 are added).

Referring simultaneously to FIG. 3, shown there generally as 315 are example relationships between anatomical features and some predetermined options 220 for a number of effects 215. As shown, the example anatomical features are in column 305, and effects 215 that may be used with media items 110 where the anatomical feature 305 are shown are provided on columns to the right of column 305. Each row in column 305 shows an example anatomical feature that may be viewable in an ultrasound media item 110 and the options 220 mapped to that anatomical feature for a given effect 215. For example, as illustrated, the “face” anatomical feature 305a is mapped to two options 220 for the image effect 215: a “Moon Mobile” option 220a and a “Baby Carriage” option 220b.

As shown in table 315, a limited number of example effects 215 (e.g., images, animations, and text) are shown for illustration purposes. However, in various embodiments, the effects 215 may include any suitable visual, audio or other effect that enhances viewing of the multimedia product to be generated. For example, additional effects 215 may include: audio, frames, borders, transitions and/or colors which can be used with ultrasound media items 110. Also, a limited number of example anatomical features are shown in column 305 of FIG. 3 for illustration purposes. However, the embodiments discussed herein may be practiced with any other anatomical feature such as brain, spine, kidney(s), liver, feet, digits, and sexual organs.

Referring back to FIG. 2, it can be seen that based on the options 220a, 220b mapped to the image effect 215 for the ultrasound media item 110b showing the “Face” anatomical feature, the “Moon Mobile” option 220a is selected for the image effect 215a that is to be used with the ultrasound media item 110b with the “Face” anatomical feature viewable. Similarly, a second image effect 215b to be used with the same media item 110b has the “Baby Carriage” option 220b selected.

Referring again to FIG. 3, it can be seen that the text “I'm cute” 220c is one of the options that is mapped to the “Face” anatomical feature 305a. Accordingly, referring back to FIG. 2, it can be seen that a text effect 215c is adapted to the media item 110b (with the “Face” anatomical feature viewable) to have the text “I'm cute” 220c.

Referring simultaneously to FIGS. 2 and 3, it can be seen that based on the options 220f, 220g, mapped to the animation effect for the “spine” anatomical feature 305e in FIG. 3, in FIG. 2, the “Flying Stork” option 220f is selected for the animation effect 215f that is to be used with the ultrasound media item 110d with the “spine” anatomical feature viewable. Similarly, a second animation effect 215g to be used with the same media item 110d has the “Heart Balloon” option 220g selected.

In FIG. 3, it can be seen that the text “Baby on Board” 220h is an option that is mapped to the “spine” anatomical feature 305e for a text effect 215. Accordingly, referring back to FIG. 2, it can be seen that a text effect 215h is adapted to the media item 110d (with the “spine” anatomical feature viewable) to have the text “Baby on Board” option 220h selected.

Referring to FIG. 4, shown there generally as 400 is an example screenshot of an ultrasound media product that have effects combined with ultrasound media items, in accordance with at least one embodiment of the present invention. The illustrated example is of a screenshot from the multimedia product between the “0:00:00” timestamp and the “0:00:05” timestamp (as shown in FIG. 2), where the effects noted above have been combined with the ultrasound media item 110b.

In the illustrated screenshot 400, it can be seen that the ultrasound media item 110b with the “Face” anatomical feature viewable has a theme applied to it, so that a border has been placed around the ultrasound media item 110b. In addition, the three effects 215 that are mapped to the “Face” anatomical feature have been adapted to the ultrasound media item 110b so as to have particular options 220 selected based on the anatomical feature viewable in the ultrasound media item 110b. As illustrated, the image effect 215a with the “Moon Mobile” option 220a selected is shown as appearing with the ultrasound media item 110b. Similarly, the image effect 215b with the “Baby Carriage” option 220b selected is shown as appearing in the same screenshot 500. Moreover, the text effect 215c is shown as being used with the text “I'm cute” option 220c selected.

Referring back to FIG. 3, it can be seen that the different options 220 can be mapped to ultrasound media items 110 that have a certain type of anatomical feature 305 viewable. In various embodiments, the options 220 may be mapped to the effects 215 based on a random matching. Additionally or alternatively, the matching may be made based on certain criteria such as an even distribution of available options 220 amongst the different available anatomical features 305.

In further embodiments, the matching may be made to associate certain options 220 with suitable anatomical features 305. For example, as shown in FIG. 3, for the “Head” anatomical feature 305b, a head-related option such as the “Bonnet” option 220d can be matched to the image effect 215. Additionally, another head-related option (e.g., the text “Helmet Hair” 220h) can be matched to the “Head” anatomical feature 305b for the text effect 215.

Referring again to FIG. 2, it can be seen that the application of the theme has configured the ultrasound media item 110c with the “Head” anatomical feature viewable to be shown between the “0:00:05” timestamp and the “0:00:10” timestamp. It can also be seen that adapting the image effect 215d to the “Head” ultrasound media item 110c has resulted in the “Bonnet” option 220d being selected for the image effect 215d. Similarly, adapting the text effect 215e to the “Head” ultrasound media item 110c has resulted in the text “Helmet Hair” option 220e being selected for the text effect 215e.

Referring to FIG. 5, shown there generally as 500 is another example screenshot of an ultrasound media product that have effects combined with ultrasound media items, in accordance with at least one embodiment of the present invention. The illustrated example is of a screenshot from the multimedia product between the “0:00:05” timestamp and the “0:00:10” timestamp (as shown in FIG. 2), where the effects noted above have been combined with the “Head” ultrasound media item 110c.

In the screenshot 500, it can be seen that the ultrasound media item 110c with the “Head” anatomical feature viewable has a theme applied to it, so that a border has been placed around the ultrasound media item 110c. In addition, the two effects 215 that are mapped to the “Head” anatomical feature shown in table 315 of FIG. 3 have been adapted to the ultrasound media item 110c. As illustrated in FIG. 5, the image effect 215d with the “Bonnet” option 220d is shown as appearing with the ultrasound media item 110c. Similarly, the text effect 215e with the “Helmet Hair” option 220e selected is also shown as appearing in the same screenshot 500.

Referring simultaneously to FIG. 5 and FIG. 1, it can be seen that the ultrasound media items 110c showing the “Head” anatomical feature appears in both figures. In FIG. 1, a BPD measurement is viewable on the ultrasound media item 110c when it forms part of a medical examination. However, in FIG. 5, because the media item 110c is being added to a multimedia product intended for viewing by a non-medical audience, the BPD measurement is removed in the illustrated example screenshot. In some situations, the purpose and location of the measurement may raise questions or cause confusion for a non-medical audience, so its removal may potentially allow for more positive reception of the multimedia product (e.g., by parents of the unborn baby). Notwithstanding, measurements do not need to be moved, and it may be possible to include ultrasound media items 110 with measurements viewable in the multimedia product.

As can be seen in FIGS. 4-5, the adapting of various effects to attributes of an ultrasound media item 110 may result in multimedia effects being displayed that make the ultrasound media items 110 more easily understandable by parents of an unborn baby. For example, as shown in FIG. 5, the appearance of the “Head” anatomical feature in the ultrasound media item 110c may not be readily apparent to parents of the unborn baby who are typically not accustomed to viewing fetal ultrasound images. However, with the addition of the image effect 215d and text effect 215e that have each been adapted to the “Head” anatomical feature so that suitable effect options 220d, 220e are selected, parents viewing the multimedia product may more readily appreciate that the ultrasound image 110c has the “Head” anatomical feature viewable.

Referring back to FIG. 3, it can be seen that for a given anatomical feature 305, there can be a variety of suitable options 220 that can be mapped to an effect 215. For example, in addition to the options 220 mapped to the various effects 215 discussed above for the “Face” anatomical feature 305a and the “Head” anatomical feature, there may be suitable options 220 mapped to effects 215 for a number of other anatomical features 305. For example, for the “Leg” anatomical feature 305c, there may be leg-related or feet-related options 220 associated with it for various effects 215. For example, as illustrated, for an image effect 215, there may be options 220j of “Socks”, “Shoes”, or “Soccer ball” matched to the effect 215; for the animation effect 215, a “Running Character” option 220k may be matched to the effect 215; and for a text effect 215, the text “Ready, set, go!” option 220l may be matched to the effect 215. Moreover, also viewable in FIG. 3 is that for a heart anatomical feature 305d, a “Heart shape” option 220l is matched to the image effect 215.

Referring back to FIG. 2, it can be seen that application of a theme to the ultrasound media items 110e, 110a, 110f may result in a configuration where each of these ultrasound media items 110 are played at the timestamps “0:00:10”, “0:00:1”, and “0:00:20” respectively. To adapt the various effects 215 to these ultrasound media items 110, it can be seen that suitable options 220 for the anatomical features viewable in each ultrasound media items 110e, 110a, 110f have been selected. For example, based on the options 220 matched to the anatomical features 305 shown table 315 of FIG. 3, a “Heart Shape” option is selected for an image effect to be used in combination with an ultrasound media item 110e with the “Heart” anatomical feature viewable. Similarly, for the ultrasound media item 110a with the “Belly” anatomical feature viewable, a “Pacifier” option is selected for the animation effect to be used with the media item 110a; and a “Bottle” option is selected for the image effect to be used with the media item 110a. Further, for the “Leg” ultrasound media item 110f, a “Socks” option is selected for an image effect and a “Running Character” option is selected for an animation effect.

Referring briefly to FIG. 3, it can be seen that “Pen” is an available option 220 for an image effect 215 to be used with both the “Arm” anatomical feature and the “Hand” anatomical feature. Depending on the nature of the ultrasound media items 110 to be included in the multimedia product, it may be possible that the same “Pen” option is selected and appears twice in a given generated multimedia product. To avoid the same effect options 220 from being repeated, in some embodiments, prior to selecting a given option 220 to be used with a given effect 215, the method may involve performing a review of whether the same option has already been selected to be used. If so, an alternative unused option 220 may be selected.

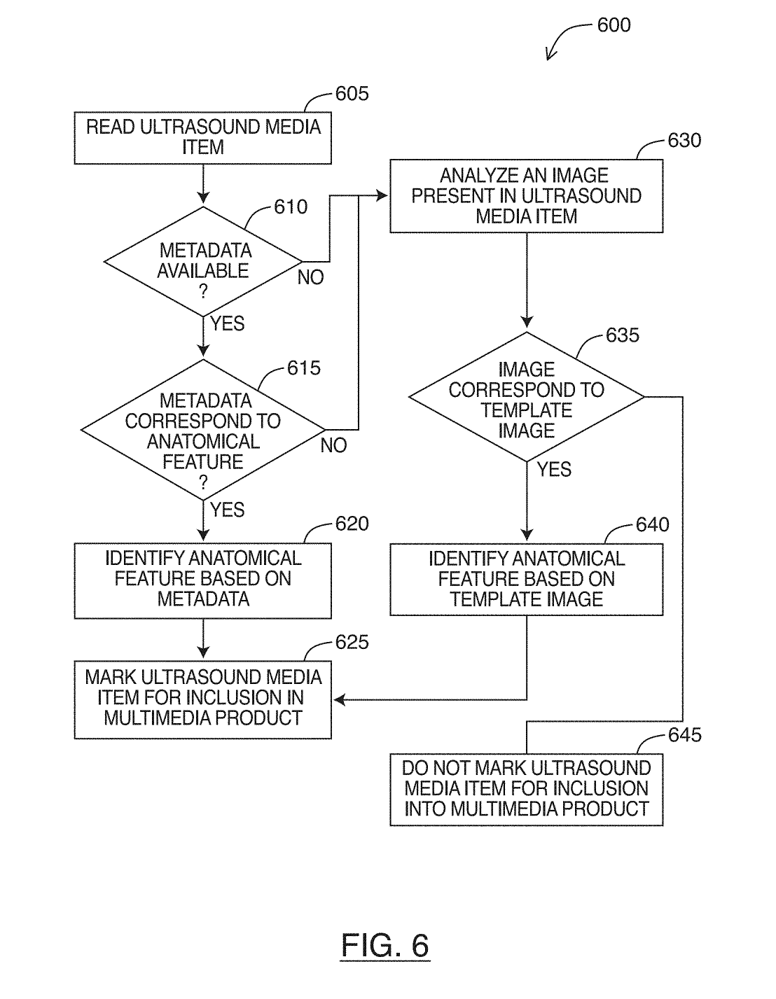

Referring to FIG. 6, shown there generally as 600 is a flowchart diagram for the steps of a method of selecting an ultrasound media item for inclusion into a multimedia product, in accordance with at least one embodiment of the present invention. As a number of the effects discussed above for applying to ultrasound media items 110 are anatomical feature-related, the method of FIG. 6 includes a number of acts related to detecting an anatomical feature viewable on at least one ultrasound media item 110. In various embodiments, the method of FIG. 6 may be performed prior to applying the theme and/or effects discussed above.

In discussing the method of FIG. 6, reference will also be made to the tables 310, 315 shown in FIG. 3. As discussed above, one embodiment of adapting an effect to an attribute of an ultrasound media item 110 may involve selecting options 220 suitable for given effects 215 based on anatomical feature(s) 305 viewable in the ultrasound media item 110. In some embodiments, the anatomical features 305 viewable within a given ultrasound media item 110 may be determined in accordance with the method of FIG. 6. Because the media items 110 have particular anatomical features viewable, they may be particularly suitable for inclusion into the multimedia product because they are more easily viewed and understood by non-medically trained individuals. Also, the media items 110 with particular anatomical features viewable may be suitable for automated adapting of various effects 215 in the manner noted above (e.g., the selection of particular options 220 for effects 215 that are suitable for given anatomical features).

At 605, the method may involve reading an ultrasound media item 110. For example, as discussed above, this may involve identifying various ultrasound media items from a medical (e.g., obstetrics) examination.

At 610, a determination may be made as to whether metadata for the ultrasound media items 110 is available. The metadata may be any information associated with the ultrasound media item 110, but is not the ultrasound media item 110 itself. For example, the metadata associated with an ultrasound media item 110 may be a measurement or an annotation made on the ultrasound media item 110.

If there is metadata available (the ‘YES’ Branch at 610), at 615, a determination may be made as to whether the metadata corresponds to an anatomical feature. For example, different types of measurements or annotations may typically be associated with respective different types of anatomical features. Referring simultaneously to FIG. 3, shown there generally as 310 are different example types of metadata 320 that may be associated with anatomical features 305. As shown in FIG. 3, table 310 shows example types of values that may be mapped to different anatomical features 305. Each row in table 310 illustrates how different values for a given item of metadata 320 can be mapped to particular anatomical features 305.

The mapping may provide a translation from common metadata values in medical examinations to the anatomical features that are typically viewable in such ultrasound media items 110. In the example table 310 of FIG. 3, there are a number of standard measurements that may be made during a regular fetal ultrasound scan. These measurements may allow a medical professional to assess the growth of the fetus in the womb. However, the measurements typically consist of specialized medical terminology or acronyms (e.g., related to internal anatomy such as bones) that is difficult for non-medically trained viewers of the generated multimedia product to understand or interpret.

For example, as illustrated in table 310 in FIG. 3, a biparietal diameter (BPD) measurement may provide the diameter across a developing baby's skull. However, a non-medically trained person would not likely know that an ultrasound media item with a BPD measurement has the head of the unborn baby viewable. By mapping the BPD measurement to the “Head” anatomical feature, that ultrasound media item 110 may, as noted above, be marked for enhancement with various head-related effects 215 and included into the multimedia product.

Generally, the mapping between types of measurements to anatomical features may provide a mechanism to extrapolate from the specialized medical terminology (e.g., indicative of internal anatomy) to external anatomical features that would be more familiar to viewers of the multimedia product being generated. As shown in table 310 of FIG. 3, a number of additional different measurements (and their associated acronyms, if applicable) are listed. For example, measurements of the humerus, radius, and/or ulna bones may be extrapolated to indicate that ultrasound media items 110 with such measurements have the arm of an unborn baby viewable. Similarly, measurements of the femur, tibia and/or fibula bones may be extrapolated to indicate that ultrasound media items 110 with such measurements have the leg of an unborn baby viewable.

The table 310 in FIG. 3 also shows another type of metadata 320 (annotations) that may be used to identify anatomical features viewable in ultrasound media items 110. For example, various phrases, keywords and/or text that are used by medical professionals may commonly appear on ultrasound media items 110 having given anatomical features viewable. Such keywords may be mapped to their corresponding anatomical features so that in act 615 of FIG. 6, the presence of any such text in the annotations of an ultrasound media item 110 may be used to determine that the corresponding anatomical feature is viewable in the ultrasound media item 110. For example, as shown in FIG. 3, the text “Profile” is mapped to the “Face” anatomical feature.

Referring back to FIG. 6, if it is determined that the metadata associated with an ultrasound media item 110 corresponds to an anatomical feature (the ‘YES’ branch at act 615), at act 620, the method may proceed to identify the anatomical feature viewable in the ultrasound media item 110 based on the metadata. This may be based on the anatomical features 305 mapped to a given type of measurement or mapped to certain text present in the annotations.

At 625, the method may proceed to mark the ultrasound media item 110 for inclusion into the multimedia product. Having identified a given ultrasound media item 110 as showing a particular anatomical feature, marking the media item 110 may allow the various anatomical feature-related effects 215 discussed above to be applied to the ultrasound media item 110.

If metadata is not available (the ‘NO’ branch at 610) or the metadata does not correspond to an anatomical feature (the ‘NO’ branch at 615—e.g., if the types of measurements or text in the annotations of an ultrasound media item 110 do not correspond to any mapped anatomical features), the method may instead proceed to act 630.

At 630, an image present in the given ultrasound media item 630 may be analyzed to determine if an anatomical feature is viewable. For example, this analysis may involve comparing the ultrasound image to a template image that is predetermined to have a given anatomical feature viewable. This may involve performing various image analysis techniques to ascertain if certain shapes or image patterns that are present in a template image for a given anatomical feature are also present in the ultrasound media item 110 being analyzed.

Referring simultaneously back to FIG. 1, the method of FIG. 6 may be performed on the various ultrasound media items 110 shown in the medical examination 100. In an example where the method of FIG. 6 is being performed to determine if any of such ultrasound media items 110 have a fetal heart viewable, when act 630 of FIG. 6 is performed, the method may attempt to determine if a given ultrasound media item 110 in the medical examination 100 corresponds to a template image of a fetal heart. As will be understood by persons skilled in the art, a common feature of fetal ultrasound images is a multiple-chamber view of the fetal heart. Accordingly, image analysis may be performed on the various ultrasound images 110 to determine if they contain a similar multiple-chamber feature visible. While image analysis of many of the ultrasound media items 110 shown in FIG. 1 would not result in a match to a standard fetal heart template image, analysis of the particular media item 110e may result in a match because of the multiple-chamber view also appearing in such media item 110e. Accordingly, the ultrasound media item 110e may be identified as an ultrasound media item 110 with a fetal heart viewable.

In other examples, template images may be provided for the various other anatomical features. A similar process of comparing given ultrasound media items 110 to the template images may be performed to determine if any such other ultrasound media items 110 match the appearance of the template images.

The template images may be provided in various ways. For example, in some embodiments, the template image may be derived from a set of pre-categorized images showing given anatomical features. These pre-categorized images may be pre-populated or seeded into a library of classified images that can be referenced during the image analysis act 630 of FIG. 6. Additionally or alternatively, as categorizations of ultrasound media items are 110 are being made from the performance of act 630 in FIG. 6, the results of such categorizations may be added to the library of classified images. In some embodiments, various additional machine learning and/or computer vision techniques may be used to help refine the template image(s) that are used during image analysis operations.

Referring still to FIG. 6, at 635, a determination can be made as to whether the analyzed image in the ultrasound media item 110 corresponds to the template image. If a given ultrasound media item 110 does correspond (e.g., it matches the template image—the ‘YES’ branch at 635), the anatomical feature viewable in the ultrasound media item 110 may be identified as the one that is associated with the template image (act 640). The ultrasound media item 110 with the determined anatomical feature may then be marked for inclusion into the multimedia product (act 625). Based on the determined anatomical feature, the various anatomical feature-related effects discussed above may then be applied to the ultrasound media item 110.

If the result of the image analysis at act 630 is that no viewable anatomical feature can be identified (the ‘NO’ branch at 635), the method may proceed to act 645. At act 645, the ultrasound media item 110 being analyzed is not marked for inclusion into the multimedia product. By performing the method of FIG. 6 to detect anatomical features prior to generating a multimedia product, the ultrasound media items 110 that are selected for inclusion into the multimedia product are more likely to be high-quality images that have clear anatomical features viewable. Additionally, as discussed above, the detection of ultrasound media items 110 with anatomical features may allow the effects 215 that are applied to be more suitable for the given ultrasound media items 110 so as to enhance their viewing by non-medically trained personnel.

In various embodiments, the methods described herein for generating a multimedia product may be implemented using a graphics library such as the three js library (which provides a JavaScript WebGL (Web Graphics Library) Application Programming Interface (API)). For example, a theme with a number of effects may be specified via a script that calls the available API calls in WebGL to set the order in which selected ultrasound media items 110 are to be played, and/or to set various visual parameters of the effects 215. In some embodiments, one or more WebGL scenes may be set up for a theme, and each scene may have an associated WebGL camera (which may be the same across one or more scenes). When applying the theme, various ultrasound media items 110 marked for inclusion into the multimedia product may be included as objects and added to a given scene. In various embodiments, if there are more ultrasound media items 110 marked for inclusion that available scenes in a theme, scenes may be reused when generating a multimedia product based on that given theme.

To add the effects 215 associated with a theme (e.g., as described above in relation to FIG. 2), various additional objects may be added to a scene containing an ultrasound media item 110 object. For example, sprites corresponding to various options 220 may be selected for the animation and image effects 215, and the placement and/or movement of the sprites may be specified using WebGL API calls. In various embodiments, the objects (e.g., sprites) added to a scene may be two-dimensional (e.g., a plane) and/or three-dimensional (e.g., with textures and geometry). In various embodiments, the position and/or rotation of these objects may be modified to create the best viewing experience for the multimedia product. In various themes, similar parameters for the camera may also be modified to create the best viewing experience for the multimedia product.

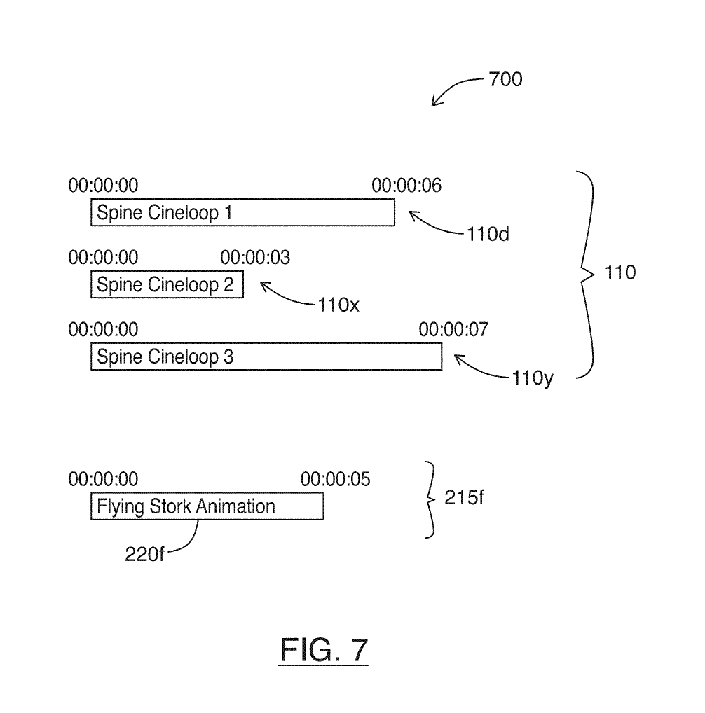

Referring to FIG. 7, shown there as 700 is an example illustration of cineloops having different lengths that are to be adapted when combined with an effect with a standard duration, in accordance with at least one embodiment of the present invention. As noted above, the application of a theme to ultrasound media items 110 may include adapting a given effect to an attribute of a media item 110, or vice versa. In some embodiments, the ultrasound media items 110 are a number of different cineloops, and the different respective attributes of the ultrasound media items 110 correspond to respective lengths of each cineloop.

Referring still to FIG. 7, an animation effect 215f may be applied to an ultrasound media item 110. However, the animation effect 215f may have a standard duration that is different from the lengths of the various cineloops to which the effect 215f may be applied. As shown in FIG. 7, several example ultrasound media items 110 (e.g., cineloops) are illustrated as horizontal bars with timestamps: a first spine cineloop 110d with a duration of 6 seconds, a second spine cineloop 110x with a duration of 3 seconds, and a third spine cineloop 110y with a duration of 7 seconds. As discussed above in relation to FIGS. 2 and 3, if it is determined that a given ultrasound media item 110 has the anatomical feature of a “spine” viewable, a “Flying Stork” option 220f may be selected for an animation effect 215f to be applied to the “Spine” cineloop 110d, 110x, 110y. However, as shown, the “Flying Stork” animation effect 215f may have a standard duration of 5 seconds. As such, some embodiments herein may involve adapting either the duration of the effect 215f to a length of a given cineloop 110d, 110x, 110y and/or adapting the length of the cineloop 110d, 110x, 110y to the duration of the effect 215f.

Various methods may be used to adapt the duration of an effect 215f to the length of a given cineloop 110d, 110x, 110y, or vice versa. In various embodiments, there may be different implementations depending on whether a cineloop length is longer than the standard duration of the effect 215f (e.g., cineloop 110d, 110y) or whether the cineloop length is shorter than the standard duration of the effect 215f (e.g., cineloop 110x).

In one example embodiment where the cineloop length is longer than the standard duration of the effect 215f, the scripting of the animation effect 215f (e.g., using WebGL) may be provided in several components: a first part that configures the sprite sliding or animating into the screen so that it can be viewed. Then, the sprite can be configured to appear as if it drifts for a period of time that depends on the length of the cineloop 110d, 110y (e.g., until the cineloop 110d, 110y is complete or almost complete). After, the visible sprites can be configured to slide off the screen (e.g., with or without the cineloop 110d, 110y itself).

In various embodiments, the drifting of the sprites (e.g., during the interim period that accounts for the unknown duration of a cineloop 110d, 110y) may be configured to look random and/or based on mathematical functions. For example, during the drifting, the positioning of the sprite on the screen may be continually modified based on a mathematical function of time (e.g., the Cartesian coordinates of the sprite may be modified according to a function for a circle such as X position=cos(t), Y position=sin(t), where ‘t’ is time). In embodiments where multiple objects are provided in a scene, the drifting paths of each object may be configured to be different. For example, this may allow each of the objects to appear independent of each other, so as to be more visually engaging. In one example embodiment, the Cartesian coordinates for each of the objects may be configured as follows: X position=sin(t/a)+sin(2t/b); Y position=sin(t/c)+sin(2t/d), where T is time, and ‘a’, ‘b’, ‘c’, and ‘d’ are random integers for each object on the screen.

In situations where the cineloop length is shorter than the standard duration of the effect (e.g., if the “Flying Stork” animation effect 215f is to be used with the second spine cineloop 110x), the cineloop length may be adapted to the standard duration of the effect 215f. For example, the animation effect 215f may be configured to have a minimum scene length that matches the standard duration of the effect 215f. In these scenarios, upon completion of a cineloop 110x, the cineloop 110x may freeze on the last screen until the animation completes. Alternatively, the cineloop 110x may loop until the animation effect 215f completes.

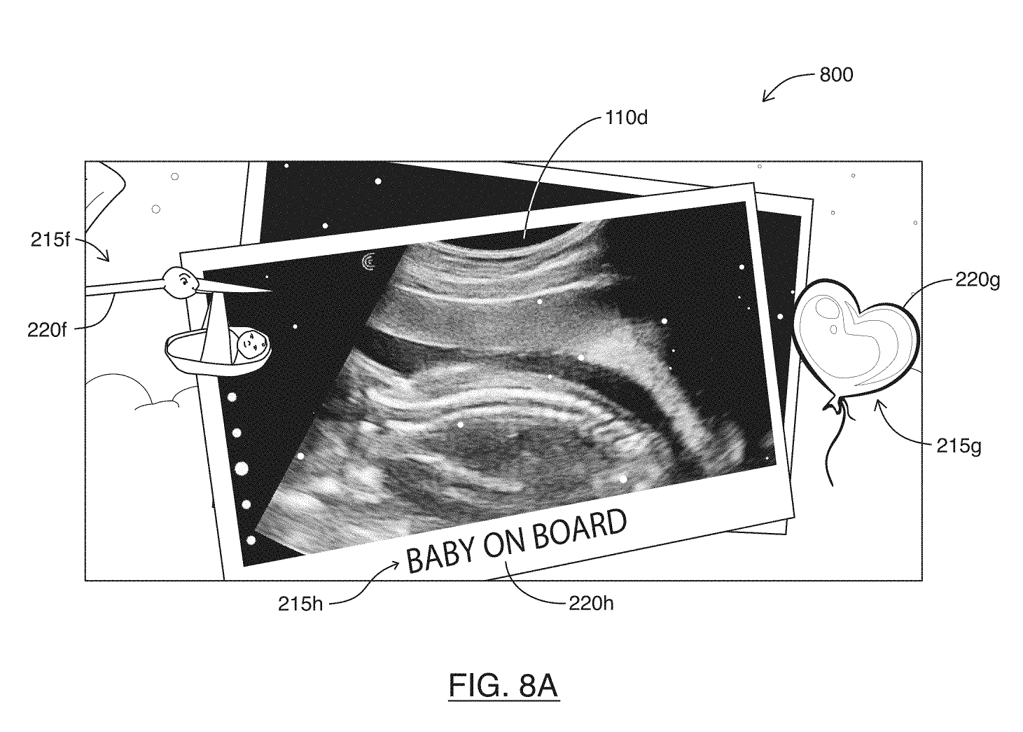

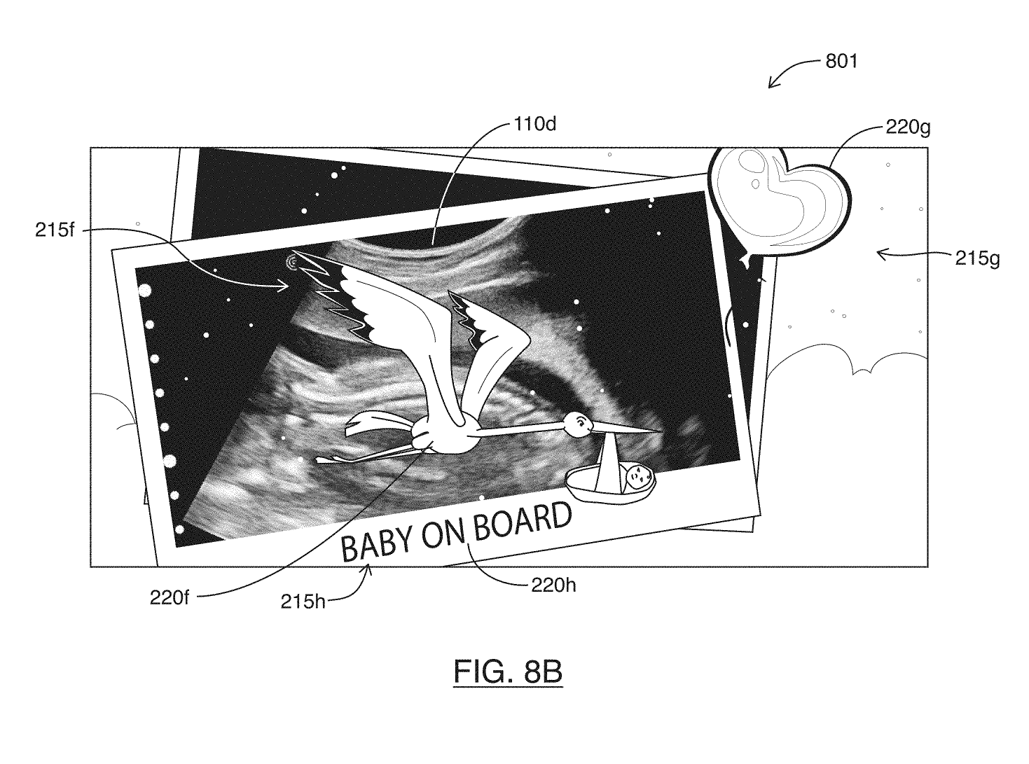

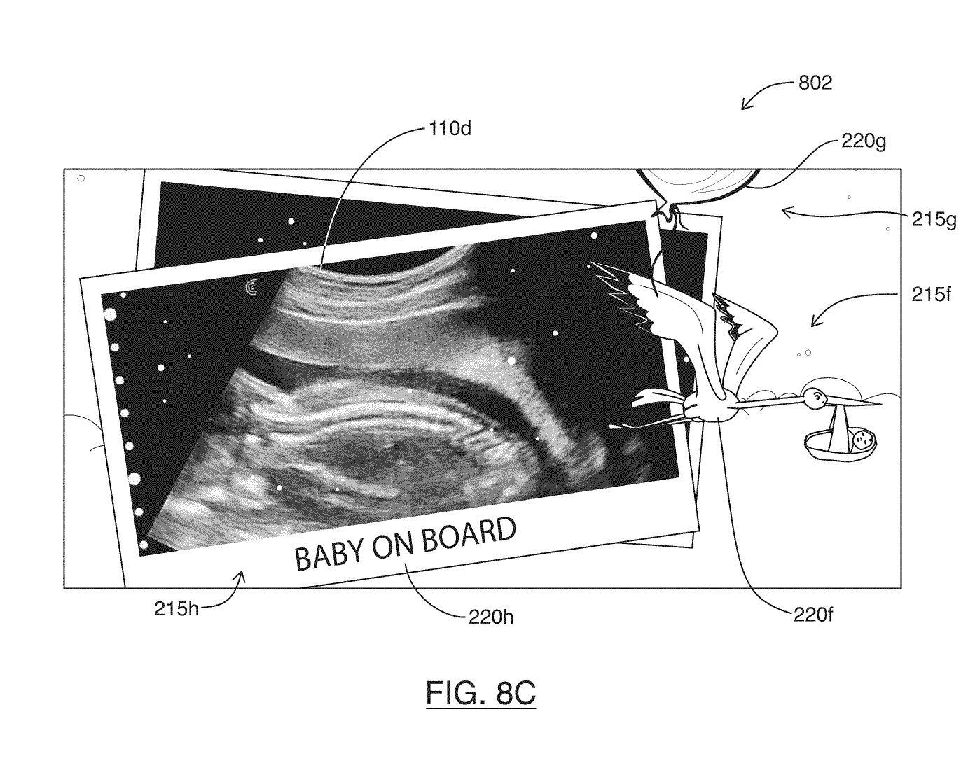

Referring to FIGS. 8A-8C, shown there generally as 800-802 are a sequence of example screenshots at various points in time of an ultrasound media product that have effects combined with ultrasound media items, in accordance with at least one embodiment of the present invention. In discussing FIGS. 8A-8C, reference will also be made to FIGS. 2 and 7. The illustrated example is of screenshots from the timeline of the multimedia product illustrated in FIG. 2, starting at the “0:00:25” timestamp, where various effects have been combined with the ultrasound media item 110d with the “spine” anatomical feature viewable. Further to the discussion above in relation to FIG. 7, FIGS. 8A-8C also illustrate how the duration of the “Flying Stork” animation effect 215f can be adapted to length of the spine cineloop 110d.

FIG. 8A shows a screenshot generally as 800 at a first point in time. The screenshot 800 is similar to that which is shown in FIGS. 4 and 5 in that the ultrasound cineloop 110d with the “spine” anatomical feature viewable has a theme applied to it, so that a border has been placed around the ultrasound media item 110d. With simultaneous reference to FIG. 2, it can be seen that the three effects 215 that are mapped to the “spine” anatomical feature has also been used with this ultrasound media item 110d. As illustrated, the animation effect 215f with the “Flying Stork” option 220f selected is shown entering the screen on the left side. Also, the animation effect 220g with the “Heart Balloon” option 220g selected is viewable on the right side of the screen. Further, the text effect 215h is shown being applied with the text “Baby on Board” 220h selected and viewable. With respect to the adapting of the duration of the animation effect 210f to the cineloop 110d, the sprite 220f for the flying stork character may enter the screen as the spine cineloop 110d begins to play.

FIG. 8B shows a screenshot generally as 801 at a second point in time. In this screenshot, it can be seen that the “Spine” ultrasound media item 110d remains viewable. However, the various animation effects 215f, 215g has progressed part way through their respective animations. In particular, it can be seen that for the first animation effect 215f with the “Flying Stork” option 220f selected, the “Stork” character has progressed from the left side of the screen (as shown in FIG. 8A) to the middle of the screen. Similarly, for the second animation effect 215g with the “Heart Balloon” option 220g selected, the “Heart Balloon” image has progressed upward from the middle of the right side of the screen (as shown in FIG. 8A) towards the top of the of the screen. Further, the text effect 215h with the mapped “Baby on Board” text 220h option selected remains viewable.

Referring simultaneously to FIG. 7, since the spine cineloop 110d has a longer length than the 5 second standard duration of the flying stork animation 215f, it can be seen in FIG. 8B that the flying stork character animation has now completed its first component by sliding into the middle of the screen. At this point, the stork character may be configured to drift (e.g., pursuant to a mathematical function for X, Y coordinates) until the cineloop 110d is complete or almost complete.

In the example screenshot shown in FIG. 8B, the path of “Stork” character is shown as overlapping with the appearance of the ultrasound media item 110d (e.g., so as to block the appearance of the ultrasound media item 110d temporarily). In some embodiments, when making the mathematical calculations of the animation path, the coordinates of the viewable area of the ultrasound media item 110d may be taken into account so as to configure the animation path to not intersect with such area. This may allow the animated character to not block the appearance of an ultrasound media item 110.

Referring to FIG. 8C, shown there generally as 802 is a screenshot at a third point in time after the screenshot of FIG. 8B. In this screenshot, it can be seen that the ultrasound media item 110d previously viewable in FIGS. 8A and 8B continues to remain viewable. However, as illustrated, the animation effects 215f, 215g that have been used with the cineloop 110d have progressed further. Particularly, the first animation 215f with the “Flying Stork” option 220f selected has progressed to the right side of the screen. Also, the second animation 215g with the “Heart Balloon” option 220g selected has similarly progressed further so that it is now mostly no longer viewable on the screen. The text effect 215h with the text “Baby on Board” option 220h selected again remains viewable in this screenshot. Referring simultaneously to FIG. 7, after the sprite for the stork character has been configured to drift until the cineloop 110d is complete or almost complete, the stork character may be configured to continue on its flight path in a direction that will eventually remove the stork character from the screen.

Referring to FIG. 9, shown there generally as 900 is a block diagram for a system of generating an ultrasound media product, in accordance with at least one embodiment of the present invention. The system 900 may include an ultrasound imaging apparatus 905, a server 930, and a viewing computing device 960; each communicably connected to network 910 (e.g., the Internet) to facilitate electronic communication.

In one example embodiment, the ultrasound imaging apparatus 905 may be provided in the form of a handheld wireless ultrasound scanner that is communicably coupled to an Internet-enabled computing device configured to transmit the ultrasound media items 110 (as shown above in relation to FIG. 1) to the server 930. In other embodiments, the ultrasound imaging apparatus 905 may be provided in the form of a unitary ultrasound machine that can scan, store, and transmit the ultrasound media items 110 from a medical examination to the server 930. In further embodiments, the ultrasound imaging apparatus 905 may simply store ultrasound media items 110 already acquired from a medical examination, and provide functionality to transmit such ultrasound media items 110 to the server 930. In various embodiments, the ultrasound imaging apparatus 905 may be configured to display a user interface 100 similar to what is shown in FIG. 1, and which provides a user-selectable option 130 that, when selected, causes the ultrasound imaging apparatus to communicate with the server 930 to cause the methods for generating a multimedia product discussed herein to be performed.

The server 930 may be configured to provide a multimedia product generator 932 to perform various acts of the methods discussed herein. The server 930 may be configured to communicate with the ultrasound imaging apparatus 905 to receive and store ultrasound media items 110 into a corresponding suitable storage mechanism such as database 934. The server 930 may also provide a multimedia product generator 932 that is configured to generate an ultrasound media product as discussed herein. For example, the multimedia product generator 932 may be configured to read ultrasound media items 110 from the corresponding database 934; mappings amongst metadata 320, anatomical features 305, and effect options 220 (e.g., as shown in tables 310, 315 of FIG. 3) stored in the anatomical feature mappings database 938; and data from the themes and effects database 936 which can store underlying data (e.g., sprites, bitmaps, and the like) that are to be used when generating a multimedia product. In various embodiments, the multimedia product generator 932 may be provided in the form of software instructions (e.g., a script) configured to execute on server 930 and/or transmitted from server 930 to a viewing computing device 960 for execution thereon. As noted above, in one example embodiment, the software instructions may be provided in the form of a script written in a scripting language that can access the WebGL API.

Although illustrated as a single server in the block diagram of FIG. 9, the term “server” herein may encompass one or more servers such as may be provided by a suitable hosted storage and/or cloud computing service. Further, in various embodiments, the databases illustrated may not reside with the server 930. For example, the data may be stored on managed storage services accessible by the server 930 and/or the viewing computing device 960 executing a script.

In some embodiments, the server 930 may also be communicably coupled to a billing or accounting system for the a medical professional associated with the ultrasound imaging apparatus 905. In such embodiments, upon generating the multimedia product, the server 930 may communicate with the billing or accounting system so as to add a charge to a patient for creation of the ultrasound multimedia product.

The viewing computing device 960 can be any suitable computing device used to generate the multimedia product and/or access the multimedia product generated by the server 930. For example, in the embodiment where the multimedia product generator 932 of server 930 is provided in the form of a script that can make WebGL API calls, the script may be transmitted to the viewing computing device 960 so that the multimedia product may be generated when the script is executed in browser 962. A graphics library (GL) engine 964 may interpret the script and live render the multimedia product for viewing at the viewing computing device 960. In some embodiments, the live render of the multimedia product may involve processing by a Graphics Processing Unit (GPU) 970 provided on the viewing computing device 960.

In some embodiments, the server 930 may be configured to execute WebGL API calls such that a script (or portion thereof) may be executed at the server 930 to perform pre-rendering. For example, this may allow for more flexible distribution of a generated multimedia product. For example, the multimedia product generator 932 may be configured to generate a standalone multimedia file (e.g., a Motion Picture Experts Group (MPEG)-4, or MP4 file) that can be transmitted from server 930 to the viewing computing device 960 for displaying thereon (e.g., for playing using a media player (not shown)). As used herein, the term “multimedia product” may refer to a pre-rendered multimedia experience (e.g., a generated video file) and/or any multimedia experience that is dynamically generated each time.

In various embodiments, the multimedia product may be configured to be interactive. For example, when a given ultrasound media item is displayed during the playback of a generated multimedia product, the multimedia product may be configured to receive user input to zoom in or otherwise highlight the ultrasound media item being displayed. Additionally or alternatively, the multimedia product may be configured to provide gallery controls on the display of various frames of the generated multimedia product. For example, these gallery controls may be configured to receive “next” or “previous” input during the display of a given ultrasound media item, so as to allow a user to advance forward to the next ultrasound media item, or navigate back to a previously-viewed ultrasound media item. In various embodiments, the interactivity may be implemented using API calls available in WebGL.

In various embodiments, the multimedia product may be configured to have an introduction screen that is displayed prior to the display of ultrasound media items. In various embodiments, the introduction screen may be configured to display text that by default is set to the patient's name entered during the medical examination. In various embodiments, the clinician may be provided with the ability to customize this text. In embodiments where the multimedia product is provided in the form of a dynamically generated video file and/or multimedia experience (e.g., from the execution of a script), the introductions screen may be displayed during the time it takes to load/transmit the script from a server 930 to a viewing computing device 960.

The various embodiments discussed herein may help automate generation of a fetal ultrasound multimedia product. Whereas traditional methods of creating an ultrasound multimedia product typically require manual identification of the media items 110 to be included, the present embodiments may help automate selection of the particular ultrasound multimedia items 110 that are suitable for inclusion (e.g., those which show particular anatomical features). Additionally, traditional ultrasound multimedia product creation methods typically require manual identification of the effects (e.g., text, animations, audio) that are suitable for the selected media items. In contrast, the present embodiments may help automate the identification of the suitable effects to be used with given ultrasound media items 110 by selecting options 220 for given effects 215 based on the anatomical features viewable in the ultrasound media items 110. Moreover, the present embodiments provide various methods of adapting a duration of effect 215 to various lengths of ultrasound media items 110, or vice versa, to further facilitate the automated creation of the multimedia product. In various embodiments, these features may be practiced individually, or by combining any two or more of the features.

As noted above, in some embodiments, the multimedia product generated from ultrasound media items 110 obtained during a regular medically-necessary examination. For example, in the case of fetal ultrasound scans, the ultrasound media items 110 may be obtained during regular obstetrics examinations where medical professionals assess the health of the unborn baby. By using ultrasound media items 110 from a medically-necessary examination to generate the multimedia product, the ALARA (As Low As Reasonably Achievable) principle can be followed with respect to avoiding unnecessary exposure to ultrasound energy.

While a number of exemplary aspects and embodiments have been discussed above, those of skill in the art will recognize that may be certain modifications, permutations, additions and sub-combinations thereof. While the above description contains many details of example embodiments, these should not be construed as essential limitations on the scope of any embodiment. Many other ramifications and variations are possible within the teachings of the various embodiments.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally include “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, digital signal processors (“DSPs”), embedded processors, graphics processors, math co-processors, mobile computers, mobile devices, tablet computers, desktop computers, server computers, cloud computers, mainframe computers, computer workstations, and the like. For example, one or more data processors in a control circuit for a device may implement methods as described herein by executing software instructions in a program memory accessible to the processors. In another example, a tablet computer or other portable computing device having a touchscreen may implement methods as described herein by having processors provided therein execute software instruction in a program memory accessible to such processors.

For example, while processes or blocks are presented in a given order herein, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

The invention may also be provided in the form of a program product. The program product may include any non-transitory medium which carries a set of computer-readable instructions which, when executed by a data processor (e.g., in a controller, ultrasound processor in an ultrasound machine, and/or a processor in an electronic display unit), cause the data processor to execute a method of the present embodiments. Program products may be in any of a wide variety of forms. The program product may include, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1.-20. (canceled)

21. A system for generating a multimedia product comprising:
a viewing computing device; and
a server, storing:
a plurality of ultrasound media items having associated metadata, the plurality of ultrasound media items being generated from a fetal ultrasound scan;
a mapping of metadata values to anatomical features of a fetus;
a mapping of the anatomical features of a fetus to options for one or more multimedia effects;
underlying data associated with the options for the one or more multimedia effects; and
a multimedia product generator comprising software instructions executable by at least one processor at the server or at the viewing computing device, wherein when the instructions are executed by the at least one processor, the at least one processor is configured to:
read metadata associated with at least one ultrasound media item of the plurality of ultrasound media items;
based on the mapping of metadata values to anatomical features of a fetus, determine an anatomical feature of a fetus, corresponding to the read metadata, that is viewable in the at least one ultrasound media item;
based on the mapping of the anatomical features of a fetus to options for the one or more multimedia effects, identify an option for a multimedia effect of the one or more multimedia effects; and
apply the multimedia effect, with the identified option, to the at least one ultrasound media item, to generate one or more frames of the multimedia product;
wherein the generated one or more frames of the multimedia product show the at least one ultrasound media item with the applied multimedia effect, and the applied multimedia effect has the option that matches the anatomical feature of a fetus determined to be viewable in the at least one ultrasound media item.

22. The system of claim 21, wherein the multimedia product generator is provided as a script that is transmittable to the viewing computing device for execution, and wherein when the script is executed at the viewing computing device, the viewing computing device generates the multimedia product.

23. The system of claim 22, wherein during display of the generated multimedia product, the multimedia product retrieves, from the server, the underlying data associated with the identified option of the applied multimedia effect.

24. The system of claim 22, wherein the script is written in a scripting language that can access a Web Graphics Library (Web GL) Application Programming Interface (API).

25. The system of claim 21, wherein the underlying data comprises one of: a bitmap and a sprite.

26. The system of claim 21, wherein the metadata comprises measurements, and the mapping of metadata values to anatomical features of a fetus maps types of measurements to the anatomical features of a fetus.

27. The system of claim 26, wherein the mapping of metadata values to anatomical features of a fetus maps multiple types of measurements to a single anatomical feature of a fetus.

28. The system of claim 21, wherein the metadata comprises annotations, and the mapping of metadata values to anatomical features of a fetus maps text in the annotations to the anatomical features of a fetus.

29. The system of claim 21, wherein the anatomical feature of a fetus determined to be viewable in the at least one ultrasound media item is selected from a group consisting of: heart, arm, leg, face, head, brain, spine, kidney, liver, sexual organ, digits, belly, feet and hand.

30. The system of claim 21, wherein the mapping of the anatomical features of a fetus to options for the multimedia effect maps a single anatomical feature of a fetus to multiple options for the multimedia effect.

31. The method of claim 21, wherein the multimedia effect is selected from the group consisting of: audio, animation, text, images, frames and borders.

32. The method of claim 21, wherein the multimedia product generator further configures the at least one processor to:
display a user interface for displaying the plurality of ultrasound media items, the user interface providing a user-selectable option for generating the multimedia product; and
receive input that selects the user-selectable option for generating the multimedia product.

33. A method of generating a multimedia product, comprising:
identifying a plurality of ultrasound media items from a fetal ultrasound scan;
reading metadata associated with at least one ultrasound media item of the plurality of ultrasound media items;
based on a mapping of metadata values to anatomical features of a fetus, determining an anatomical feature of a fetus, corresponding to the read metadata, that is viewable in the at least one ultrasound media item;
based on a mapping of the anatomical features of a fetus to options for a multimedia effect, identifying an option for the multimedia effect; and
applying the multimedia effect, with the identified option, to the at least one ultrasound media item, to generate one or more frames of the multimedia product;
wherein the generated one or more frames of the multimedia product show the at least one ultrasound media item with the applied multimedia effect, and the applied multimedia effect has the option that matches the anatomical feature of a fetus determined to be viewable in the at least one ultrasound media item.

34. The method of claim 33, wherein the metadata comprises measurements, and the mapping of metadata values to anatomical features of a fetus maps types of measurements to the anatomical features of a fetus.

35. The method of claim 34, wherein the mapping of metadata values to anatomical features of a fetus maps multiple types of measurements to a single anatomical feature of a fetus.

36. The method of claim 33, wherein the metadata comprises annotations, and the mapping of metadata values to anatomical features of a fetus maps text in the annotations to the anatomical features of a fetus.

37. The method of claim 33, wherein the anatomical feature of a fetus determined to be viewable in the at least one ultrasound media item is selected from a group consisting of: heart, arm, leg, face, head, brain, spine, kidney, liver, sexual organ, digits, belly, feet and hand.

38. The method of claim 33, wherein the mapping of the anatomical features of a fetus to options for the multimedia effect maps a single anatomical feature of a fetus to multiple options for the multimedia effect.

39. The method of claim 33, wherein the multimedia effect is selected from the group consisting of: audio, animation, text, images, frames and borders.

40. The method of claim 33, wherein prior to the identifying the plurality of ultrasound media items, the method further comprises:
displaying a user interface for displaying the plurality of ultrasound media items, the user interface providing a user-selectable option for generating the multimedia product; and
receiving input that selects the user-selectable option for generating the multimedia product.
